(19) United States
(12) Patent Application Publication
(10) Pub. No.: US 2026/0330791 A1
(43) Pub. Date: Oct. 22, 2026

(54) Image Processing Apparatus, Image Processing System, Image Processing Method, and Image Processing Program

(71) Applicants: TERUMO KABUSHIKI KAISHA, Tokyo (JP); ROKKEN, INC., Osaka (JP)

(72) Inventors: Shunsuke YOSHIZAWA, Ebina-shi (JP); Yasukazu SAKAMOTO, Sakai-city (JP); Clément JACQUET, Sakai-city (JP); Stephen TCHEN, Sakai-city (JP); Kaan ERASLAN, Sakai-city (JP); Maxime LENFANT, Sakai-city (JP)

(73) Assignees: TERUMO KABUSHIKI KAISHA, Tokyo (JP); ROKKEN, INC., Osaka (JP)

(21) Appl. No.: 19/578,992

(22) Filed: Mar. 26, 2026

Related U.S. Application Data
(63) Continuation of application No. PCT/JP2024/034284, filed on Sep. 25, 2024.

(30) Foreign Application Priority Data
Sep. 29, 2023 (JP) 2023169236

(51) Int. Cl. G06T 19/20; A61B 6/00; A61B 6/46; A61B 6/50; G06T 7/73

(52) U.S. Cl. CPC G06T 19/20; A61B 6/466; A61B 6/487; A61B 6/504; A61B 6/5211; G06T 7/73; G06T 2200/04; G06T 2207/10121; G06T 2207/10132; G06T 2207/30021; G06T 2207/30108; G06T 2207/30244; G06T 2210/41; G06T 2219/2021

(57) ABSTRACT

An image processing apparatus that generates a three-dimensional image by imaging a three-dimensional structure of a biological lumen based on a plurality of two-dimensional images obtained in a manner that a sensor provided in a catheter inserted into the biological lumen moves in a section from a first point to a second point in the biological lumen along an axis of the catheter includes a control unit that adjusts a shape of the three-dimensional image in accordance with a shape of the axis of the catheter in the section, the shape of the axis of the catheter being specified by using at least one X-ray image including an image of at least a part of the catheter imaged by an X-ray imaging device that performs fluoroscopy of the biological lumen.

### CROSS-REFERENCES TO RELATED APPLICATIONS

This application is a continuation of International Application No. PCT/JP2024/034284 filed on Sep. 25, 2024, which claims priority to Japanese Patent Application No. 2023-169236 filed on Sep. 29, 2023, the entire content of both of which is incorporated herein by reference.

### TECHNOLOGICAL FIELD

The present disclosure relates to an image processing apparatus, an image processing system, an image processing method, and an image processing program.

### BACKGROUND DISCUSSION

International Patent Application Publication WO 2022/045182 A1 discloses a method for specifying a ventral direction of a patient in a three-dimensional image of a biological tissue when the three-dimensional image is generated and displayed based on a two-dimensional ultrasonic image acquired by a transducer of an ultrasonic catheter.

At the time of catheter surgery such as a stent graft indwelling procedure, in order to reduce X-ray exposure or assist a catheter operation, it is conceivable to merge a three-dimensional computed tomography (CT) image obtained by imaging before surgery or a digital subtraction angiography (DSA) image obtained by imaging during surgery with an X-ray image generated in real time and display the merged image. A DSA image is obtained by subtracting an X-ray image that serves as a mask and is obtained by fluoroscopy of a blood vessel before administration of a contrast agent from a contrast image obtained by fluoroscopy of the blood vessel after administration of the contrast agent. By merging the three-dimensional CT image or the DSA image with the X-ray image, the detailed shape of the blood vessel and the real-time state can be simultaneously confirmed. However, when a hard guidewire or catheter enters into the blood vessel, the blood vessel may be stretched, and a large gap between the three-dimensional CT image and the actual organ shape may be generated. X-ray exposure during imaging of a three-dimensional CT image may also be a problem. In the DSA image, since a large amount of contrast agent is used, contrast agent nephropathy may occur.

Therefore, instead of displaying a three-dimensional CT image obtained by imaging before surgery, it is conceivable to display a three-dimensional image generated by a method without X-ray exposure as disclosed in International Patent Application Publication WO 2022/045182 A1 during surgery. However, in a case where the blood vessel meanders, the shape of the blood vessel may not be able to be reproduced by a three-dimensional image simply by stacking two-dimensional ultrasonic images.

### SUMMARY

The present disclosure facilitates reproduction of a shape of a biological lumen with a three-dimensional image when the three-dimensional image is generated by imaging the three-dimensional structure of the biological lumen.

Some aspects of the present disclosure are described below.
[1] An image processing apparatus that generates a three-dimensional image by imaging a three-dimensional structure of a biological lumen based on a plurality of two-dimensional images obtained in a manner that a sensor provided in a catheter inserted into the biological lumen moves in a section from a first point to a second point in the biological lumen along an axis of the catheter, the image processing apparatus including: a control unit that adjusts a shape of the three-dimensional image in accordance with a shape of the axis of the catheter in the section, the shape of the axis of the catheter being specified by using at least one X-ray image including an image of at least a part of the catheter imaged by an X-ray imaging device that performs fluoroscopy of the biological lumen.
[2] The image processing apparatus according to [1], in which the control unit specifies a position of the sensor as a sensor position for each two-dimensional image included in the plurality of two-dimensional images, sets, in the three-dimensional image, a moving line extending in a shape corresponding to the shape of the axis of the catheter in the section from a position in the three-dimensional image of a sensor position specified for a two-dimensional image obtained by the sensor when the sensor exists at the first point, and adjusts a shape of the three-dimensional image by arranging each specified sensor position on the set moving line.
[3] The image processing apparatus according to [2], in which the control unit associates a first position corresponding to the first point on the at least one X-ray image with a position of one end of the moving line, associates a second position corresponding to the second point on the at least one X-ray image with a position of another end of the moving line, sets a linking line linking the first position and the second position on the at least one X-ray image, and sets a shape of the moving line according to an operation of manually changing the set shape of the linking line.
[4] The image processing apparatus according to [2], in which the control unit associates a first position corresponding to the first point on the at least one X-ray image with a position of one end of the moving line, associates a second position corresponding to the second point on the at least one X-ray image with a position of another end of the moving line, and sets a shape of the moving line to be a shape corresponding to a linking line linking the first position, the second position, and at least one position different from the first position and the second position, according to an operation of manually designating the at least one position on the at least one X-ray image.
[5] The image processing apparatus according to [2], in which the control unit associates a first position corresponding to the first point on the at least one X-ray image with a position of one end of the moving line, associates a second position corresponding to the second point on the at least one X-ray image with a position of another end of the moving line, automatically detects an image of the axis of the catheter from the at least one X-ray image, and sets a shape of the moving line based on the detected image.
[6] The image processing apparatus according to [2], in which the control unit associates a first position corresponding to the first point on the at least one X-ray image with a position of one end of the moving line, associates a second position corresponding to the second point on the at least one X-ray image with a position of another end of the moving line, automatically detects an image of the sensor from each X-ray image included in a plurality of X-ray images imaged by the X-ray imaging device while the sensor moves in the section, and sets a shape of the moving line based on the detected image.
[7] The image processing apparatus according to any one of [2] to [6], in which the control unit associates a first position corresponding to the first point on the at least one X-ray image with a position of one end of the moving line according to an operation of manually designating the first position when the sensor exists at the first point.
[8] The image processing apparatus according to [7], in which the control unit associates a second position corresponding to the second point on the at least one X-ray image with a position of another end of the moving line according to an operation of manually designating the second position when the sensor exists at the second point.
[9] The image processing apparatus according to [7], in which the control unit acquires ratio information indicating a ratio of a dimension of the at least one X-ray image with respect to a dimension of the three-dimensional image, and associates a position separated from the first position by a distance corresponding to a distance of the moving line on the at least one X-ray image with a position of another end of the moving line as a second position corresponding to the second point, on the at least one X-ray image based on the acquired ratio information.
[10] The image processing apparatus according to any one of [2] to [6], in which the control unit automatically detects a first position corresponding to the first point on the at least one X-ray image when the sensor exists at the first point and associates the first position with a position of one end of the moving line.
[11] The image processing apparatus according to [10], in which the control unit automatically detects a second position corresponding to the second point on the at least one X-ray image when the sensor exists at the second point and associates the second position with a position of another end of the moving line.
[12] The image processing apparatus according to any one of [2] to [11], in which the control unit causes a display to display the three-dimensional image with being superimposed on the at least one X-ray image.
[13] The image processing apparatus according to [12], in which the control unit updates the three-dimensional image according to a change in the moving line during setting of the moving line.
[14] An image processing system including: the image processing apparatus according to any one of [1] to [13]; and a display that displays the three-dimensional image and the at least one X-ray image.
[15] An image processing method of generating a three-dimensional image by imaging a three-dimensional structure of a biological lumen based on a plurality of two-dimensional images obtained in a manner that a sensor provided in a catheter inserted into the biological lumen moves in a section from a first point to a second point in the biological lumen along an axis of the catheter, the image processing method including: by a control unit, adjusting a shape of the three-dimensional image in accordance with a shape of the axis of the catheter in the section, the shape of the axis of the catheter being specified by using at least one X-ray image including an image of at least a part of the catheter imaged by an X-ray imaging device that performs fluoroscopy of the biological lumen.
[16] An image processing program for causing a computer that generates a three-dimensional image by imaging a three-dimensional structure of a biological lumen based on a plurality of two-dimensional images obtained in a manner that a sensor provided in a catheter inserted into the biological lumen moves in a section from a first point to a second point in the biological lumen along an axis of the catheter to execute an operation including adjusting a shape of the three-dimensional image in accordance with a shape of the axis of the catheter in the section, the shape of the axis of the catheter being specified by using at least one X-ray image including an image of at least a part of the catheter imaged by an X-ray imaging device that performs fluoroscopy of the biological lumen.

According to the present disclosure, when a three-dimensional image is generated by imaging a three-dimensional structure of a biological lumen, a shape of the biological lumen can be easily reproduced with the three-dimensional image.

### BRIEF DESCRIPTION OF THE DRAWINGS

FIG. 1 is a block diagram illustrating a configuration of an image processing system according to an embodiment of the present disclosure.

FIG. 2 is a flowchart illustrating an operation of an image processing apparatus according to the embodiment of the present disclosure.

FIG. 3 is a diagram illustrating an example of a screen displayed on a display according to the embodiment of the present disclosure.

FIG. 4 is a diagram illustrating an example of update of a screen displayed on the display according to the embodiment of the present disclosure, by step S4.

FIG. 5 is a diagram illustrating an example of a three-dimensional image generated by the image processing apparatus according to the embodiment of the present disclosure.

FIG. 6 is a diagram illustrating an example of shape adjustment of a three-dimensional image generated by the image processing apparatus according to the embodiment of the present disclosure, by step S7.

FIG. 7 is a diagram illustrating an example of update of a screen displayed on the display according to the embodiment of the present disclosure, by Step S7.

FIG. 8 is a flowchart illustrating a first example of a detailed procedure of Step S6.

FIG. 9 is a diagram illustrating an example of update of a screen displayed on the display according to the embodiment of the present disclosure, by step S603.

FIG. 10 is a diagram illustrating an example of update of a screen displayed on the display according to the embodiment of the present disclosure, by step S604.

FIG. 11 is a flowchart illustrating a second example of the detailed procedure of step S6.

FIG. 12 is a flowchart illustrating a third example of the detailed procedure of Step S6.

FIG. 13 is a diagram illustrating an example of update of a screen displayed on the display according to the embodiment of the present disclosure, by Step S623.

FIG. 14 is a flowchart illustrating a fourth example of the detailed procedure of Step S6.

FIG. 15 is a flowchart illustrating a fifth example of the detailed procedure of Step S6.

FIG. 16 is a flowchart illustrating a modification example of the operation of the image processing apparatus according to the embodiment of the present disclosure.

### DETAILED DESCRIPTION

Hereinafter, an embodiment of the present disclosure will be described with reference to the drawings.

In the drawings, the same or corresponding parts are denoted by the same reference numerals. In the description of the present embodiment, the description of the same or corresponding parts will be omitted or simplified as appropriate.

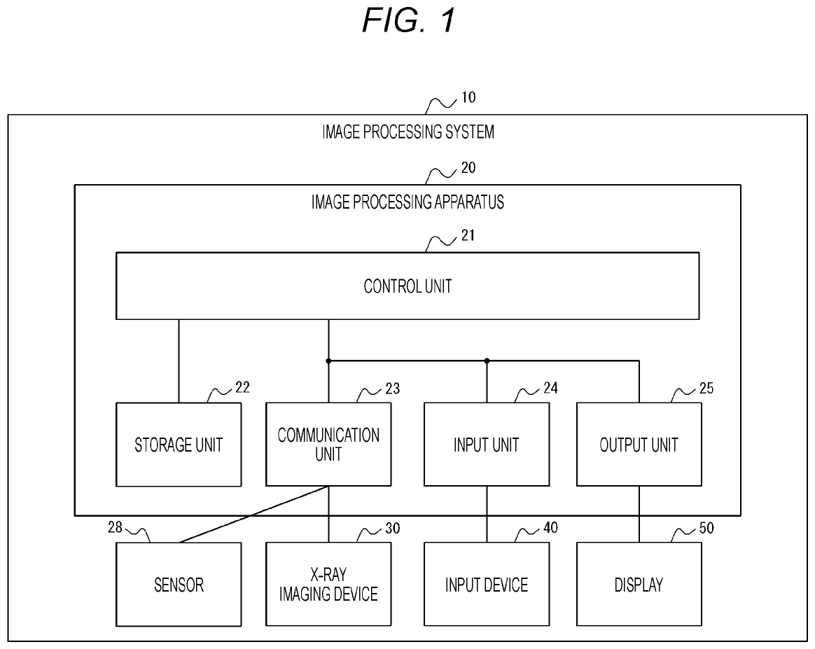

A configuration of an image processing system 10 according to the present embodiment will be described with reference to FIG. 1.

The image processing system 10 includes an image processing apparatus 20, a sensor 28, an X-ray imaging device 30, an input device 40, and a display 50. The image processing apparatus 20 is connected to the sensor 28, the X-ray imaging device 30, the input device 40, and the display 50 via a cable or a network, or in a wireless manner.

The image processing apparatus 20 can be, for example, a general-purpose computer such as a personal computer (PC), a server computer such as a cloud server, or a dedicated computer. The image processing apparatus 20 may be installed in a medical facility such as a hospital or may be installed in a facility such as a data center, which is different from the medical facility.

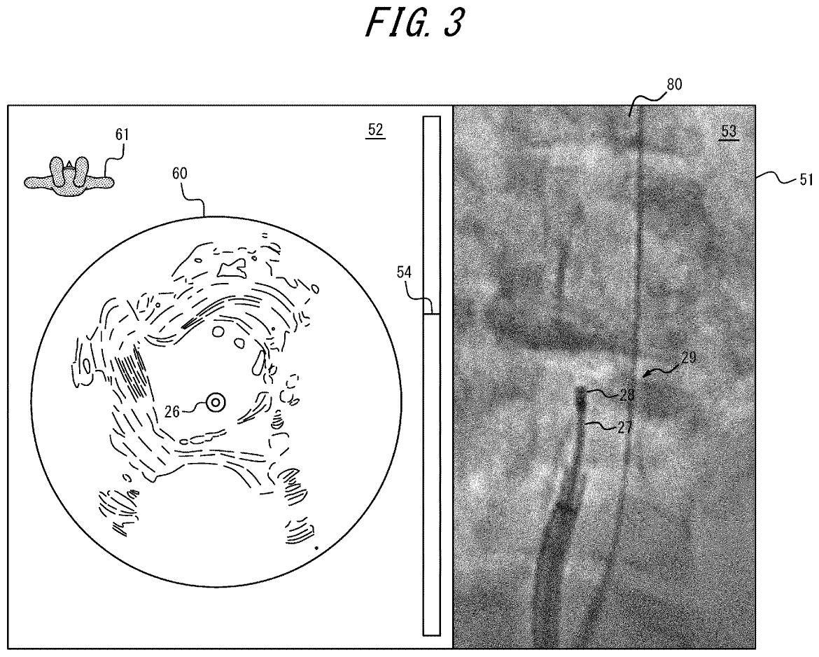

The sensor 28 can be, for example, an ultrasonic transducer used in intravascular ultrasound (IVUS). The sensor 28 transmits an ultrasonic wave while moving along an axis 27 of a catheter 26 inserted into a biological lumen such as a blood vessel as illustrated in FIG. 3 and receives a reflected wave of the transmitted ultrasonic wave.

The X-ray imaging device 30 can be, for example, a C-arm. The X-ray imaging device 30 is installed in a medical facility and performs fluoroscopy of a biological lumen.

The input device 40 can be, for example, a pointing device such as a mouse, a keyboard, or a touch screen provided integrally with the display 50. The input device 40 is installed in a medical facility and is used by an operator such as a doctor or a clinical engineer for an operation of controlling display of various types of information including an image on the display 50.

The display 50 can be, for example, a liquid crystal display (LCD) or an organic electro luminescent (EL) display. The display 50 can be installed in a medical facility and displays various types of information including an image for supporting an operator in catheter surgery such as a stent graft indwelling procedure.

A configuration of the image processing apparatus 20 according to the present embodiment will be described with reference to FIG. 1.

The image processing apparatus 20 includes a control unit 21, a storage unit 22, a communication unit 23, an input unit 24, and an output unit 25.

The control unit 21 includes at least one processor, at least one programmable circuit, at least one dedicated circuit, or any combination of the at least one processor, the at least one programmable circuit, and the least one dedicated circuit. The processor is a general-purpose processor such as a central processing unit (CPU) or a graphics processing unit (GPU), or a dedicated processor specialized for specific processing. The programmable circuit can be, for example, a field-programmable gate array (FPGA). The dedicated circuit can be, for example, an application specific integrated circuit (ASIC). The control unit 21 executes processing related to the operation of the image processing apparatus 20 while controlling each unit of the image processing apparatus 20.

The storage unit 22 includes at least one semiconductor memory, at least one magnetic memory, at least one optical memory, or any combination the at least one semiconductor memory, the at least one magnetic, and the least one optical memory. The semiconductor memory can be, for example, a random access memory (RAM), a read only memory (ROM), or a flash memory. The RAM can be, for example, a static random access memory (SRAM) or a dynamic random access memory (DRAM). The ROM can be, for example, an electrically erasable programmable read only memory (EEPROM). The flash memory can be, for example, a solid-state drive (SSD). The magnetic memory can be, for example, a hard disk drive (HDD). The storage unit 22 can function as, for example, a main storage device, an auxiliary storage device, or a cache memory. The storage unit 22 stores data to be used for the operation of the image processing apparatus 20 and data obtained by the operation of the image processing apparatus 20.

The communication unit 23 includes at least one communication module. The communication module can be, for example, a module compatible with a wired LAN communication standard such as Ethernet® or a wireless LAN communication standard such as IEEE 802.11 (i.e. Institute of Electrical and Electronics Engineers 802.11). The communication unit 23 receives data used for the operation of the image processing apparatus 20 and transmits data obtained by the operation of the image processing apparatus 20. The communication unit 23 is connected to the sensor 28 and the X-ray imaging device 30. Note that the communication unit 23 does not need to be the communication module as described above as long as an image signal can be received from the sensor 28 and the X-ray imaging device 30.

The input unit 24 includes at least one input interface. The input interface can be, for example, a universal serial bus (USB) interface, High-Definition Multimedia Interface (HDMI®) interface, or an interface compatible with short-range wireless communication standards such as Bluetooth®. The input unit 24 receives an operation of inputting data used for the operation of the image processing apparatus 20. The input unit 24 is connected to the input device 40.

The output unit 25 includes at least one output interface. The output interface can be, for example, a USB interface, an HDMI interface, or an interface compatible with a short-range wireless communication standard such as Bluetooth. The output unit 25 outputs data obtained by the operation of the image processing apparatus 20. The output unit 25 is connected to the display 50.

A function of the image processing apparatus 20 is implemented by executing an image processing program according to the present embodiment by the processor as the control unit 21. That is, the function of the image processing apparatus 20 is implemented by software. The image processing program causes a computer to function as the image processing apparatus 20 by causing the computer to execute the operation of the image processing apparatus 20. That is, the computer functions as the image processing apparatus 20 by executing the operation of the image processing apparatus 20 in accordance with the image processing program.

The program can be stored in a non-transitory computer-readable medium. The non-transitory computer-readable medium can be, for example, a flash memory, a magnetic recording device, an optical disc, a magneto-optical recording medium, or a ROM. The distribution of the program can be carried out, for example, by selling, transferring, or lending a portable medium such as a Secure Digital (SD) card, a digital versatile disc (DVD), or a compact disc read only memory (CD-ROM) that stores the program. The program may be distributed by storing the program in a storage of a server and transferring the program from the server to another computer. The program may be provided as a program product.

The computer temporarily stores, for example, the program stored in the portable medium or the program transferred from the server in the main storage device. The computer causes the processor to read the program stored in the main storage device and execute processing in accordance with the read program. The computer may read the program directly from the portable medium and execute the processing in accordance with the program. Each time the program is transferred from the server to the computer, the computer may sequentially execute processing in accordance with the received program. The processing may be executed by a so-called ASP-type service (i.e., application service provider-type service) that does not transfer the program from the server to the computer but implements the function solely through execution instructions and result acquisition. The program includes information that is intended for use in processing by an electronic computer and is equivalent to the program. For example, data that is not a direct command to the computer but has a property that defines processing of the computer corresponds to “data equivalent to the program”.

Some or all of the functions of the image processing apparatus 20 may be implemented by a programmable circuit or a dedicated circuit as the control unit 21. That is, some or all of the functions of the image processing apparatus 20 may be implemented by hardware.

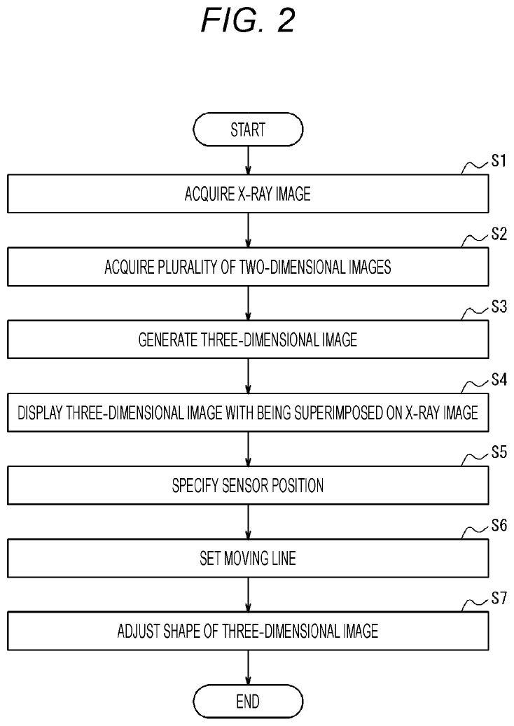

The operation of the image processing apparatus 20 according to the present embodiment will be described with reference to FIG. 2. The operation described below corresponds to an image processing method according to the present embodiment. That is, the image processing method according to the present embodiment includes steps S1 to S7 illustrated in FIG. 2.

In S1, the control unit 21 acquires one or more X-ray images imaged by the X-ray imaging device 30. Specifically, the control unit 21 receives one or more X-ray images from the X-ray imaging device 30 via the communication unit 23.

In S2, the control unit 21 acquires a plurality of two-dimensional images obtained by the sensor 28 while the sensor 28 moves in a section from a first point P1 to a second point P2 in a biological lumen. Specifically, the control unit 21 receives from the sensor 28 via the communication unit 23, reflected wave information regarding a reflected wave received by the sensor 28 while the sensor 28 moves in the section from the first point P1 to the second point P2 in the biological lumen. The control unit 21 generates a plurality of cross-sectional images as a plurality of two-dimensional images by imaging the cross-sectional structure of the biological lumen based on the reflected wave information received. Alternatively, the control unit 21 may receive from an IVUS device via the communication unit 23, a plurality of cross-sectional images generated based on the reflected wave information by the IVUS device that is separately provided, as a plurality of two-dimensional images.

Step S1 may be executed before S2, or may be executed during S2, or may be executed after S2. The control unit 21 causes the display 50 to display a screen 51 as illustrated in FIG. 3 via the output unit 25 at the latest until both steps S1 and S2 are completed.

The screen 51 includes a first region 52 and a second region 53 adjacent to the first region 52. In the example illustrated in FIG. 3, the second region 53 is arranged on the right side of the first region 52, and the second region 53 may be arranged on the left side of the first region 52.

In the first region 52, a latest two-dimensional image 60 and a human body model image 61 among the plurality of two-dimensional images acquired in S2 are displayed. The human body model image 61 is displayed such that a direction of the human body model image 61 is aligned with that of the two-dimensional image 60. In the example illustrated in FIG. 3, it is assumed that the catheter 26 is inserted from the base of the leg of a surgery subject, the upper side of the two-dimensional image 60 corresponds to the side where the abdomen of the subject exists, and the lower side of the two-dimensional image 60 corresponds to the side where the back of the subject exists. Therefore, the human body model image 61 is displayed such that the bottom surface is visible, the abdomen is upward, and the back is downward. In the example illustrated in FIG. 3, the human body model image 61 is arranged at the upper left of the two-dimensional image 60, and the human body model image 61 may be arranged at any position as long as it is in the vicinity of the two-dimensional image 60. The arrangement of the human body model image 61 may be fixed or may be freely changeable.

A slider 54 is further displayed in the first region 52. The slider 54 indicates the position of the sensor 28 in the long axis direction of the catheter 26. The slider 54 is arranged at an end of the first region 52 closer to the second region 53. Therefore, in the example illustrated in FIG. 3, the slider 54 is arranged at the right end of the first region 52.

An X-ray image 80 acquired in S1 is displayed in the second region 53. The X-ray image 80 is imaged by the X-ray imaging device 30 when the axis 27 of the catheter 26 exists at least over the section from the first point P1 to the second point P2 in the biological lumen. The X-ray image 80 includes an image of at least a part of the catheter 26, specifically an image of the axis 27 of the catheter 26 and an image of the sensor 28. In the example illustrated in FIG. 3, the X-ray image 80 further includes an image of a guidewire 29.

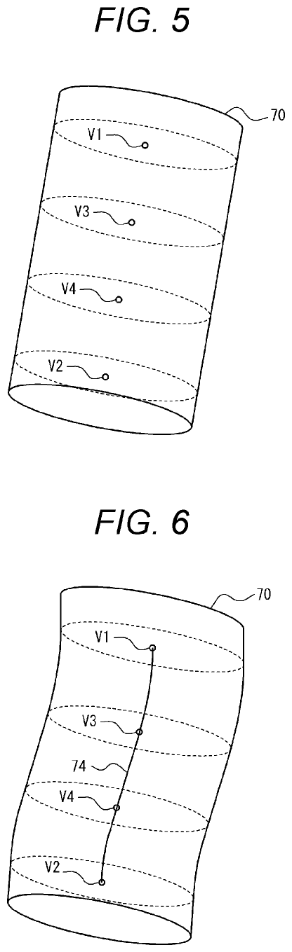

In S3, the control unit 21 images the three-dimensional structure of the biological lumen based on the plurality of two-dimensional images acquired in S2 and generates a three-dimensional image 70 as illustrated in FIG. 5. Specifically, the control unit 21 generates the three-dimensional image 70 by stacking the plurality of cross-sectional images generated or received in S2.

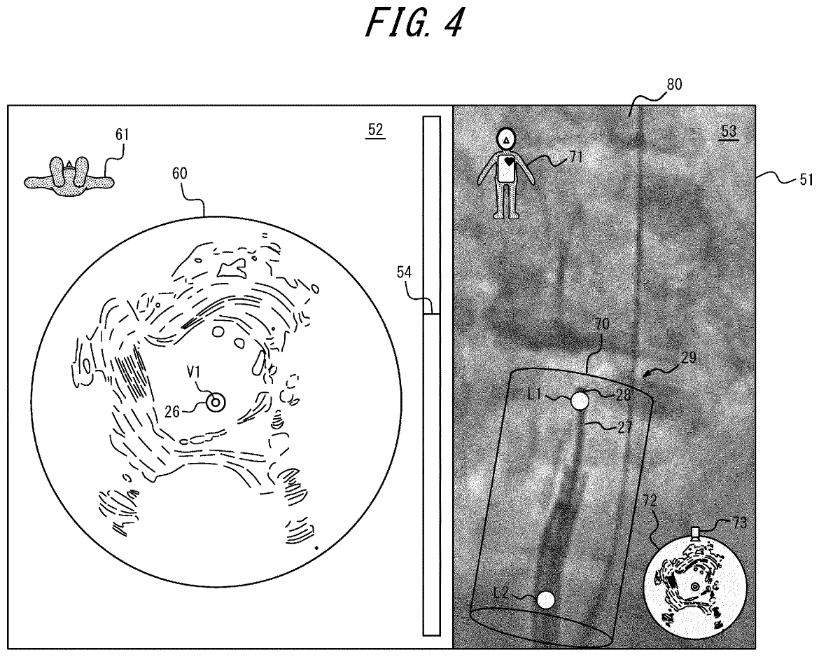

In S4, the control unit 21 causes the display 50 to display the three-dimensional image 70 generated in S3 with being superimposed on the X-ray image 80 acquired in S1. For example, as illustrated in FIG. 4, the control unit 21 updates the screen 51 displayed on the display 50 via the output unit 25.

In the second region 53, the three-dimensional image 70 generated in S3 and a human body model image 71 are displayed with being superimposed on the X-ray image 80 acquired in S1. In the example illustrated in FIG. 4, it is assumed that the angle of the three-dimensional image 70 corresponds to the angle of the surgery subject viewed from the front, the upper side of the three-dimensional image 70 corresponds to the side on which the head of the surgery subject exists, and the lower side of the three-dimensional image 70 corresponds to the side on which the foot of the subject exists. Therefore, the human body model image 71 is displayed such that the front can be viewed, and the head is directed upward, and the foot is directed downward. In the example illustrated in FIG. 4, the human body model image 71 is arranged at the upper left of the three-dimensional image 70, and the human body model image 71 may be arranged at any position as long as the human body model image 71 is in the vicinity of the three-dimensional image 70. The arrangement of the human body model image 71 may be fixed or may be freely changeable. The three-dimensional image 70 may be partially cut along the long axis direction of the catheter 26 and the cut part may be hidden so that the internal structure of the biological lumen can be observed.

In the second region 53, a cross-sectional image 72 is further displayed. The cross-sectional image 72 is obtained by reducing the same image as the two-dimensional image 60 displayed in the first region 52. A camera icon 73 indicating the position of a viewpoint is arranged at an edge of the cross-sectional image 72. Therefore, it can be relatively easy to understand from which direction the three-dimensional image 70 displayed in the second region 53 is viewed in the cross-sectional image 72.

An image display direction in which the three-dimensional image 70 is displayed on the display 50 is manually or automatically adjusted to a direction viewed from a viewpoint corresponding to an imaging angle at which the X-ray image 80 is imaged by the X-ray imaging device 30. The relative position and size of the three-dimensional image 70 displayed on the display 50 with respect to the X-ray image 80 have already been adjusted in the example illustrated in FIG. 4, but do not need to be actually adjusted yet. FIG. 4 illustrates a first position L1 corresponding to the first point P1 and a second position L2 corresponding to the second point P2 on the X-ray image 80, and the first position L1 and the second position L2 may be manually designated or automatically detected until S6.

In S5, the control unit 21 specifies the position of the sensor 28 as the sensor position for each two-dimensional image included in the plurality of two-dimensional images acquired in S2. Specifically, the control unit 21 specifies, as a first sensor position V1, the position of the center of the two-dimensional image 60 obtained by the sensor 28 when the sensor 28 exists at the first point P1. The control unit 21 specifies, as a second sensor position V2, the position of the center of the two-dimensional image 60 obtained by the sensor 28 when the sensor 28 exists at the second point P2. The control unit 21 specifies the positions of the center of the two-dimensional image 60 obtained by the sensor 28 when the sensor 28 is present at a third point P3 and a fourth point P4 different from the first point P1 and the second point P2, as a third sensor position V3 and a fourth sensor position V4, respectively. The control unit 21 may further specify, as the sensor position, the position of the center of the two-dimensional image 60 obtained by the sensor 28 when the sensor 28 is present at a point different from the first point P1, the second point P2, the third point P3, and the fourth point P4. Alternatively, the control unit 21 does not need to specify the fourth sensor position V4.

In S6, the control unit 21 sets a moving line 74 as illustrated in FIG. 6 in the three-dimensional image 70 generated in S3. The moving line 74 is a line extending in a shape corresponding to the shape of the axis 27 of the catheter 26 in the section from the first point P1 to the second point P2 in the biological lumen, from the position of the first sensor position V1 specified in S5 in the three-dimensional image 70. The shape of the axis 27 of the catheter 26 in the section from the first point P1 to the second point P2 in the biological lumen is specified by using at least one X-ray image acquired in S1. Specifically, the shape of the axis 27 of the catheter 26 from the first position L1 to the second position L2 on the X-ray image 80 acquired in S1 is specified as the shape of the axis 27 of the catheter 26 in the section from the first point P1 to the second point P2 in the biological lumen.

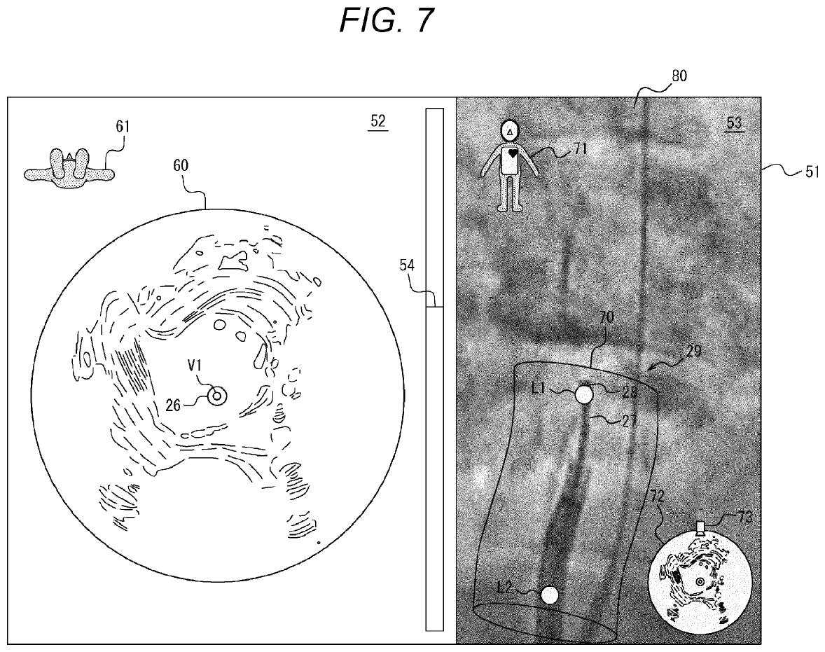

In S7, the control unit 21 adjusts the shape of the three-dimensional image 70 generated in S3 in accordance with the shape of the axis 27 of the catheter 26 in the section from the first point P1 to the second point P2 in the biological lumen specified by using at least one X-ray image. Specifically, as illustrated in FIG. 6, the control unit 21 adjusts the shape of the three-dimensional image 70 generated in S3 by arranging each sensor position specified in S5 on the moving line 74 set in S6. For example, as illustrated in FIG. 7, the control unit 21 updates the screen 51 displayed on the display 50 via the output unit 25.

Step S7 may be executed after S6 and is desirably executed simultaneously with S6. That is, during the setting of the moving line 74, the control unit 21 desirably updates the three-dimensional image 70 according to the change of the moving line 74.

In order to reduce the amount of calculation when adjusting the shape of the three-dimensional image 70 in S7, the control unit 21 desirably converts voxel data of the three-dimensional image 70 into triangular or quadrangular mesh data when generating the three-dimensional image 70 in S3. As a method of converting voxel data into mesh data, any method such as a Marching cubes method, Surface Nets, or Delaunay triangulation can be used. As a method of causing the mesh data to deform when adjusting the shape of the three-dimensional image 70, any method such as rotation minimizing frames (RMF) or Frenet-Serret expression can be used.

As a modification example of the present embodiment, the control unit 21 may cause the display 50 to separately display the three-dimensional image 70 and the X-ray image 80 in S4. As another modification example, the control unit 21 may further acquire another X-ray image imaged by the X-ray imaging device 30 at an angle different from that of the X-ray image 80 in S1 and further adjust the shape of the three-dimensional image 70 in a depth direction by using the other X-ray image in S4 to S7.

As described above, in the present embodiment, the control unit 21 generates the three-dimensional image 70 by imaging the three-dimensional structure of the biological lumen based on a plurality of two-dimensional images 60 obtained in a manner that the sensor 28 provided in the catheter 26 inserted into the biological lumen moves in the section from the first point P1 to the second point P2 in the biological lumen along the axis 27 of the catheter 26. The control unit 21 adjusts the shape of the three-dimensional image 70 in accordance with the shape of the axis 27 of the catheter 26 in the section from the first point P1 to the second point P2 in a biological lumen, which has been specified by using at least one X-ray image including the image of the sensor 28 imaged by the X-ray imaging device 30 that performs fluoroscopy of the biological lumen.

Therefore, according to the present embodiment, when the three-dimensional image 70 is generated by imaging the three-dimensional structure of the biological lumen, the shape of the biological lumen is easily reproduced by the three-dimensional image 70. For example, even if the blood vessel meanders, the shape of the blood vessel can be easily reproduced with the three-dimensional image 70.

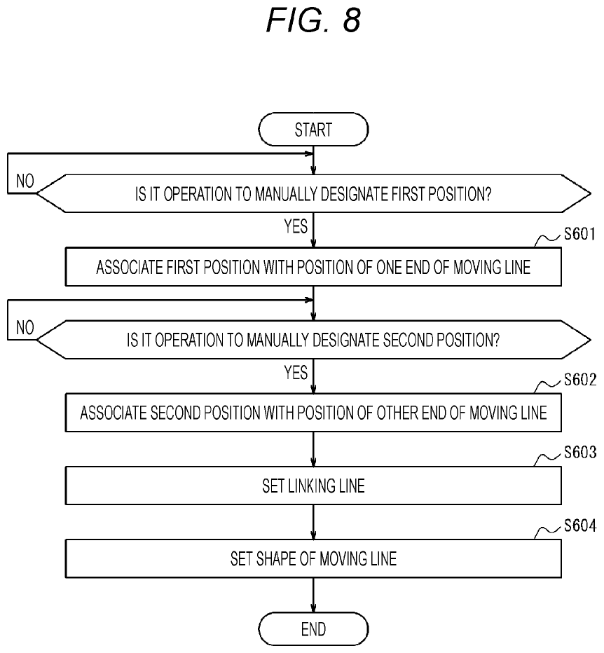

A first example of a detailed procedure of step S6 will be described with reference to FIG. 8.

In S601, the control unit 21 associates the first position L1 with the position of one end of the moving line 74 according to the operation of manually designating the first position L1 when the sensor 28 exists at the first point P1. Specifically, the control unit 21 receives, via the input unit 24, an operation of the input device 40 clicking or touching a position where the sensor 28 appears on the X-ray image imaged by the X-ray imaging device 30 when the sensor 28 exists at the first point P1. The control unit 21 receives from a motor drive unit (MDU) that drives the catheter 26 via the communication unit 23, a notification of the position of the sensor 28 in the long axis direction of the catheter 26 when the sensor 28 exists at the first point P1. The control unit 21 specifies the first sensor position V1 specified in S5 in the cross section existing at the position notified from the MDU in the three-dimensional image 70 as the position of the first sensor position V1 in the three-dimensional image 70. The control unit 21 causes the storage unit 22 to store the clicked or touched position as the first position L1, and the position of the first sensor position V1 in the three-dimensional image 70 as the position of one end of the moving line 74 in association with each other.

In S602, the control unit 21 associates the second position L2 with the position of the other end of the moving line 74 according to an operation of manually designating the second position L2 when the sensor 28 exists at the second point P2. Specifically, the control unit 21 receives, via the input unit 24, an operation of the input device 40 clicking or touching a position where the sensor 28 appears on the X-ray image imaged by the X-ray imaging device 30 when the sensor 28 exists at the second point P2. The control unit 21 receives from the MDU that drives the catheter 26 via the communication unit 23, a notification of the position of the sensor 28 in the long axis direction of the catheter 26 when the sensor 28 exists at the second point P2. The control unit 21 specifies the second sensor position V2 specified in S5 in the cross section existing at the position notified from the MDU in the three-dimensional image 70, as the position of the second sensor position V2 in the three-dimensional image 70. The control unit 21 causes the storage unit 22 to store the clicked or touched position as the second position L2, and the position of the second sensor position V2 in the three-dimensional image 70 as the position of the other end of the moving line 74 in association with each other.

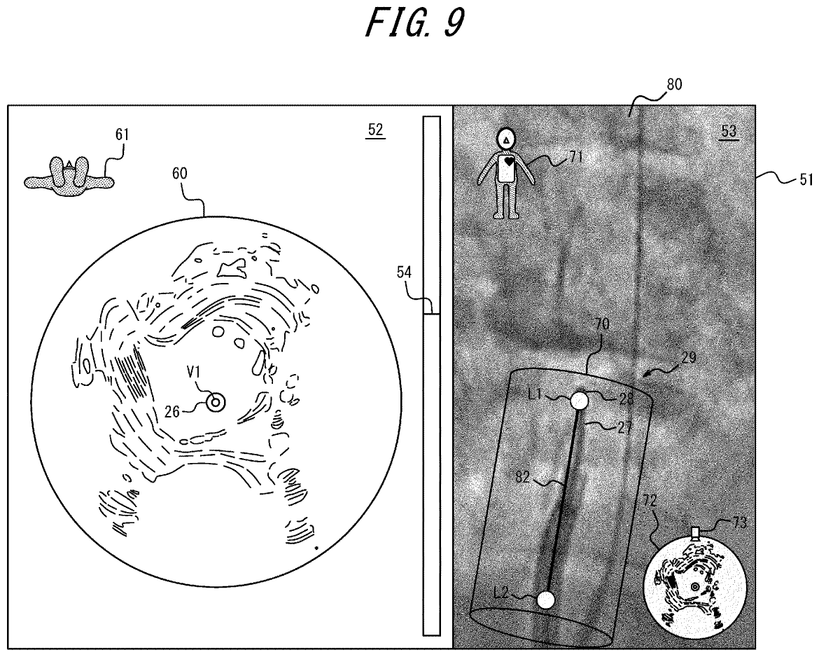

In S603, the control unit 21 sets a linking line 82 as illustrated in FIG. 9 on the X-ray image 80. The linking line 82 is a line linking the first position L1 and the second position L2. For example, as illustrated in FIG. 9, the control unit 21 arranges a mark such as a circle at each of the first position L1 and the second position L2 on the X-ray image 80 displayed on the screen 51, and draws a straight line linking the arranged marks as the linking line 82. In the example illustrated in FIG. 9, the X-ray image 80 is imaged when the sensor 28 exists at the first point P1 but may be imaged at any time point as long as the X-ray image is imaged when the axis 27 of the catheter 26 is present at least over a section from the first point P1 to the second point P2 in the biological lumen.

The control unit 21 adjusts the relative position and size of the three-dimensional image 70 displayed on the display 50 with respect to the X-ray image 80 such that the position and length of the moving line 74 coincide with the position and length of the linking line 82. Specifically, the control unit 21 adjusts the relative position and size at which the three-dimensional image 70 is displayed on the display 50 with respect to the X-ray image 80 such that the positions of both ends of the moving line 74, which have been stored in the storage unit 22, coincide with the first position L1 and the second position L2, which have been stored in the storage unit 22 in association with them.

In S604, the control unit 21 sets the shape of the moving line 74 according to the operation of manually changing the shape of the linking line 82 set in S603. Specifically, the control unit 21 receives, via the input unit 24, an operation of bending the linking line 82 by dragging any position of the linking line 82 other than the first position L1 and the second position L2 on the X-ray image 80 displayed on the screen 51 using the input device 40. The control unit 21 adjusts the shape of the moving line 74 to coincide with the bent shape of the linking line 82 each time the linking line 82 is bent.

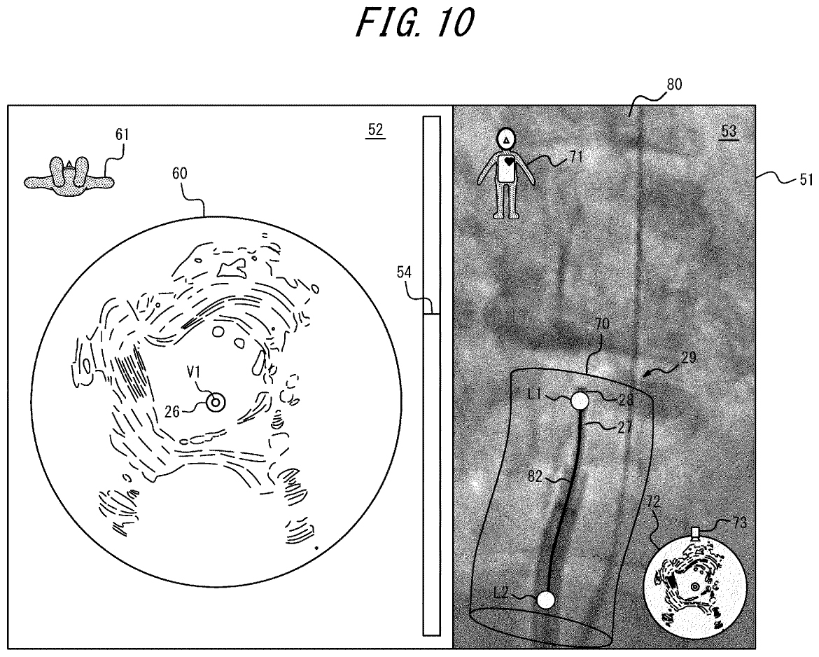

After S604, when Step S7 is executed, for example, the control unit 21 updates the screen 51 displayed on the display 50 via the output unit 25 as illustrated in FIG. 10.

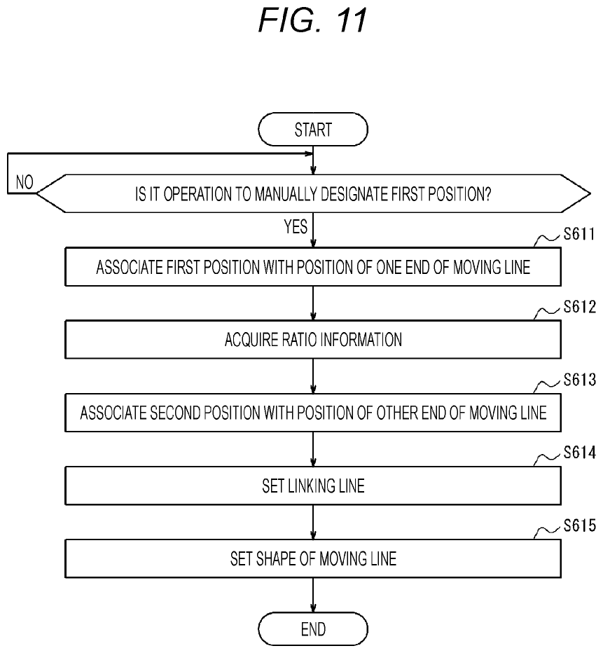

A second example of the detailed procedure of Step S6 will be described with reference to FIG. 11.

Since step S611 is similar to step S601 illustrated in FIG. 8, description of step S611 will be omitted.

In Step S612, the control unit 21 acquires ratio information indicating the ratio of the dimension of the X-ray image 80 with respect to the dimension of the three-dimensional image 70. Specifically, when the actual scale of the X-ray image 80 is known, the control unit 21 calculates the ratio of a distance on the X-ray image 80 with respect to the moving distance of the sensor 28 as the ratio information.

In S613, the control unit 21 associates a position separated from the first position L1 by a distance corresponding to the distance of the moving line 74 with the position of the other end of the moving line 74 as the second position L2 on the X-ray image 80 based on the ratio information acquired in S612. Specifically, the control unit 21 receives from the MDU that drives the catheter 26 via the communication unit 23, a notification of the position of the sensor 28 in the long axis direction of the catheter 26 when the sensor 28 exists at the second point P2. The control unit 21 specifies the second sensor position V2 specified in S5 in the cross section existing at the position notified from the MDU in the three-dimensional image 70, as the position of the second sensor position V2 in the three-dimensional image 70. The control unit 21 calculates the moving distance when the sensor 28 moves in the section from the first point P1 to the second point P2, based on the information notified from the MDU. The control unit 21 converts the calculated moving distance into the distance on the X-ray image 80 according to the ratio calculated in S612. The control unit 21 causes the storage unit 22 to store the positions separated from the first position L1 designated in S611 by the converted distance as the second position L2, and the position of the second sensor position V2 in the three-dimensional image 70 as the position of the other end of the moving line 74 in association with each other. A direction of the second position L2 with respect to the first position L1 may be designated manually or may be automatically specified in accordance with the position of the axis 27 of the catheter 26 on the X-ray image 80.

Steps S614 and S615 are similar to steps S603 and S604 illustrated in FIG. 8, respectively, and thus description of steps S614 and S615 will be omitted.

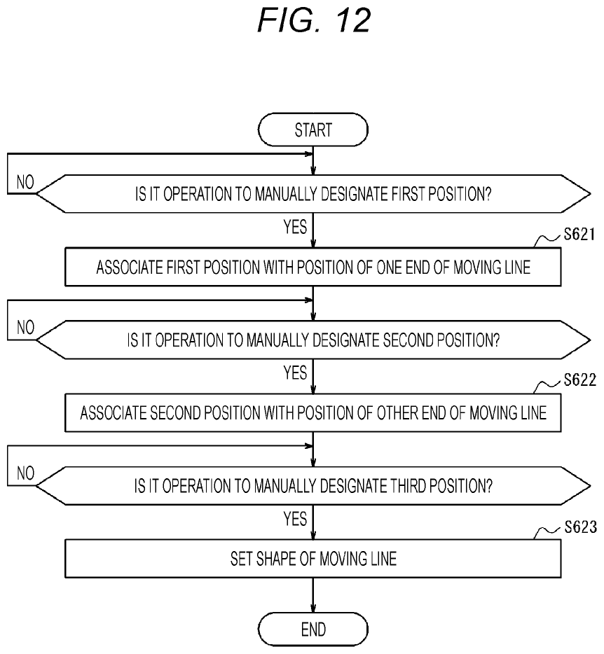

A third example of the detailed procedure of step S6 will be described with reference to FIG. 12.

Steps S621 and S622 are similar to steps S601 and S602 illustrated in FIG. 8, respectively, and thus description steps S621 and S622 will be omitted. Step S622 may be replaced with steps S612 and S613 illustrated in FIG. 11. In such a modification example, an operation of manually designating the second position L2 is not necessary.

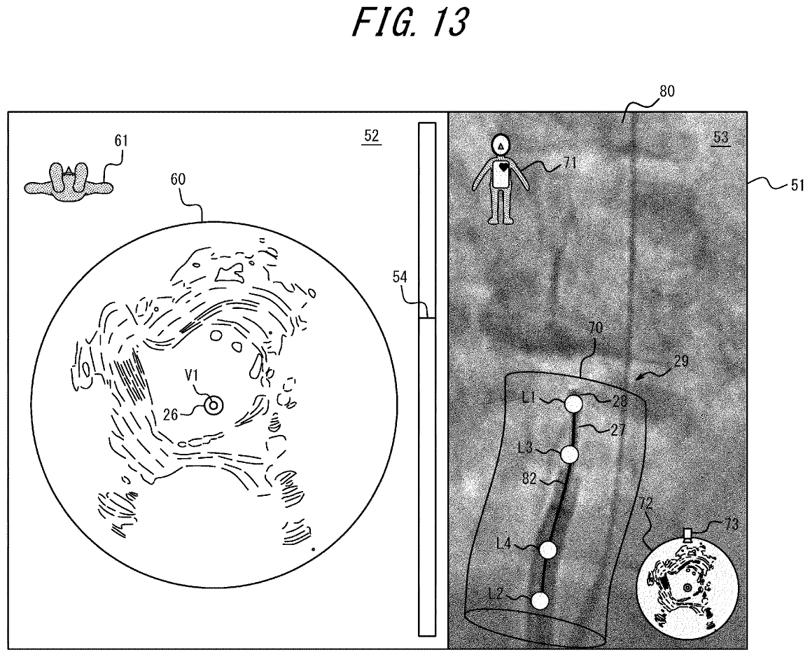

In S623, the control unit 21 sets the shape of the moving line 74 to the shape corresponding to the linking line 82 linking the first position L1, the second position L2, and the at least one position according to the operation of manually designating the at least one position different from the first position L1 and the second position L2 on the X-ray image 80. Specifically, the control unit 21 receives an operation of clicking or touching any position other than the first position L1 and the second position L2 of the axis 27 of the catheter 26, as a third position L3, by the input device 40 on the X-ray image 80 displayed on the screen 51 via the input unit 24. The control unit 21 further receives an operation of clicking or touching any position other than the first position L1, the second position L2, and the third position L3 of the axis 27 of the catheter 26, as a fourth position L4, by the input device 40 on the X-ray image 80 displayed on the screen 51 via the input unit 24. For example, as illustrated in FIG. 13, the control unit 21 arranges a mark such as a circle at each of the first position L1, the second position L2, the third position L3, and the fourth position L4 on the X-ray image 80 displayed on the screen 51, and draws a straight line linking all the arranged marks as the linking line 82. The control unit 21 may receive, via the input unit 24, an operation of clicking or touching any position other than the first position L1, the second position L2, the third position L3, and the fourth position L4 of the axis 27 of the catheter 26 by the input device 40 on the X-ray image 80 displayed on the screen 51. Alternatively, the control unit 21 does not need to receive an operation of clicking or touching the fourth position L4.

The control unit 21 adjusts the relative position and size of the three-dimensional image 70 displayed on the display 50 with respect to the X-ray image 80 such that the position and length of the moving line 74 coincide with the position and length of the linking line 82. Specifically, the control unit 21 adjusts the relative position and size at which the three-dimensional image 70 is displayed on the display 50 with respect to the X-ray image 80 such that the positions of both ends of the moving line 74, which have been stored in the storage unit 22, coincide with the first position L1 and the second position L2, which have been stored in the storage unit 22 in association with them.

Every time the position other than the first position L1 and the second position L2 is designated, the control unit 21 automatically changes the shape of the linking line 82 and changes the shape of the moving line 74 in accordance with the change. Specifically, every time the position other than the first position L1 and the second position L2 is clicked or touched, the control unit 21 newly arranges a mark such as a circle at the clicked or touched position and updates the linking line 82 to pass through the newly arranged mark on the X-ray image 80 displayed on the screen 51. Then, the control unit 21 adjusts the shape of the moving line 74 to coincide with the updated shape of the linking line 82.

After S623, when step S7 is executed, for example, the control unit 21 updates the screen 51 displayed on the display 50 via the output unit 25 as illustrated in FIG. 13.

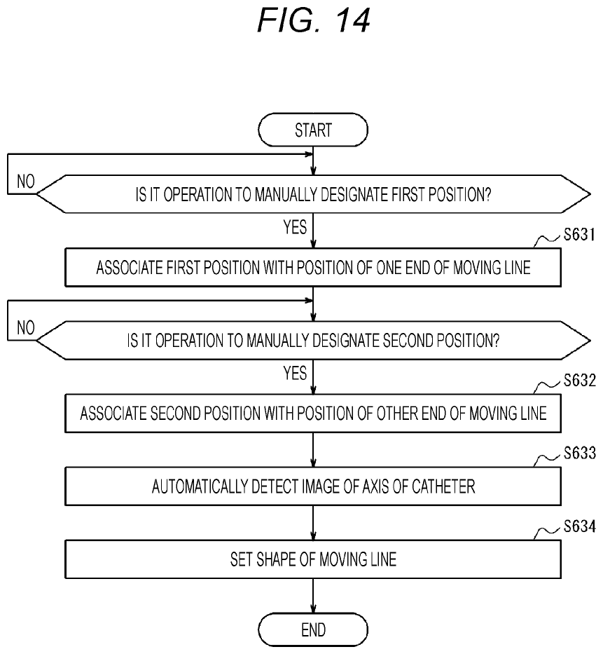

A fourth example of the detailed procedure of step S6 will be described with reference to FIG. 14.

Steps S631 and S632 are similar to steps S601 and S602 illustrated in FIG. 8, respectively, and thus description steps S631 and S632 will be omitted. Step S632 may be replaced with steps S612 and S613 illustrated in FIG. 11. In such a modification example, an operation of manually designating the second position L2 is not necessary.

In S633, the control unit 21 automatically detects the image of the axis 27 of the catheter 26 from the X-ray image 80. As a method of detecting the image of the axis 27 of the catheter 26, a known image recognition technique can be used. Machine learning such as deep learning may be used.

In S634, the control unit 21 sets the shape of the moving line 74 based on the image detected in S633. Specifically, the control unit 21 adjusts the relative position and size at which the three-dimensional image 70 is displayed on the display 50 with respect to the X-ray image 80 such that the positions of both ends of the moving line 74, which have been stored in the storage unit 22, coincide with the first position L1 and the second position L2, which have been stored in the storage unit 22 in association with them. Then, the control unit 21 adjusts the shape of the moving line 74 to coincide with the shape of the image detected in S633.

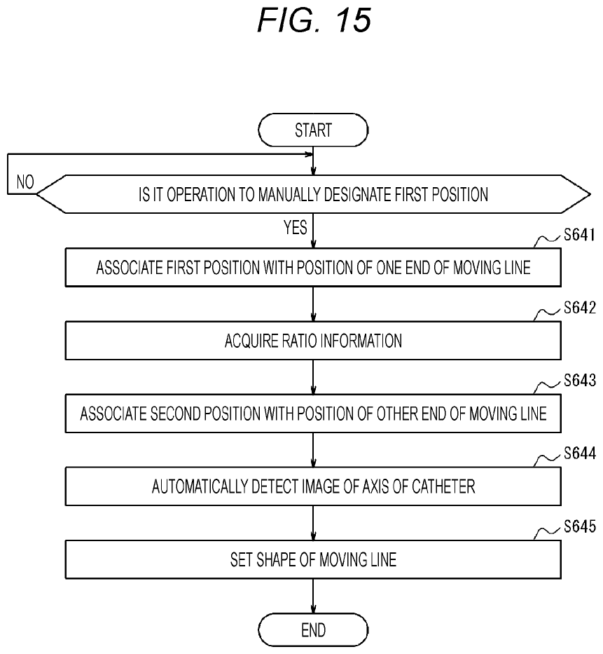

A fifth example of the detailed procedure of step S6 will be described with reference to FIG. 15.

This example is obtained by replacing step S632 with steps S612 and S613 illustrated in FIG. 11 in the fourth example. Since step S641 is similar to step S601 illustrated in FIG. 8, description of step 641 will be omitted. Steps S642 and S643 are similar to steps S612 and S613 illustrated in FIG. 11, respectively, and thus description of steps S642 and S643 will be omitted. Steps S644 and S645 are similar to steps S633 and S634 illustrated in FIG. 14, respectively, and thus description steps S633 and S634 will be omitted.

In the first example to the fifth example, instead of receiving the operation of manually designating the first position L1 when the sensor 28 exists at the first point P1, the control unit 21 may automatically detect the first position L1 when the sensor 28 exists at the first point P1 and associate the first position L1 with the position of one end of the moving line 74. In such a modification example, the control unit 21 automatically detects the position where the sensor 28 appears on the X-ray image imaged by the X-ray imaging device 30 when the sensor 28 exists at the first point P1. As a method of detecting the position where the sensor 28 appears, a known image recognition technique can be used. Machine learning such as deep learning may be used. The control unit 21 receives from an MDU that drives the catheter 26 via the communication unit 23, a notification of the position of the sensor 28 in the long axis direction of the catheter 26 when the sensor 28 exists at the first point P1. The control unit 21 specifies the first sensor position V1 specified in S5 in the cross section existing at the position notified from the MDU in the three-dimensional image 70 as the position of the first sensor position V1 in the three-dimensional image 70. The control unit 21 causes the storage unit 22 to store the automatically detected position as the first position L1, and the position of the first sensor position V1 in the three-dimensional image 70 as the position of one end of the moving line 74 in association with each other.

In the first example, the third example, and the fifth example, instead of receiving the operation of manually designating the second position L2 when the sensor 28 exists at the second point P2, the control unit 21 may automatically detect the second position L2 when the sensor 28 exists at the second point P2 and associate the second position L2 with the position of the other end of the moving line 74. In such a modification example, the control unit 21 automatically detects the position where the sensor 28 appears on the X-ray image imaged by the X-ray imaging device 30 when the sensor 28 exists at the second point P2. As a method of detecting the position where the sensor 28 appears, a known image recognition technique can be used. Machine learning such as deep learning may be used. The control unit 21 receives from the MDU that drives the catheter 26 via the communication unit 23, a notification of the position of the sensor 28 in the long axis direction of the catheter 26 when the sensor 28 exists at the second point P2. The control unit 21 specifies the second sensor position V2 specified in S5 in the cross section existing at the position notified from the MDU in the three-dimensional image 70, as the position of the second sensor position V2 in the three-dimensional image 70. The control unit 21 causes the storage unit 22 to store the automatically detected position as the second position L2, and the position of the second sensor position V2 in the three-dimensional image 70 as the position of the other end of the moving line 74 in association with each other.

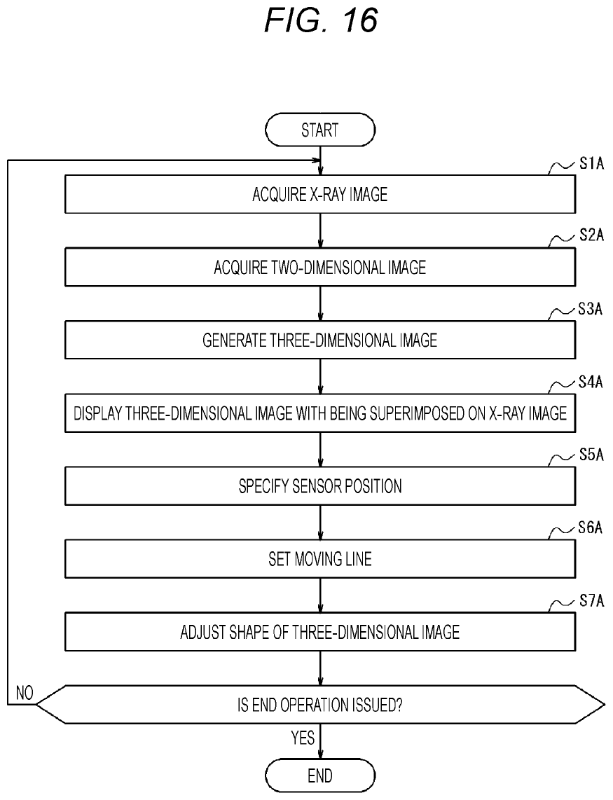

A modification example of the operation illustrated in FIG. 2 will be described with reference to FIG. 16. Regarding each of steps S1A to S7A illustrated in FIG. 16, description of processing similar to steps S1 to S7 illustrated in FIG. 2 will be omitted or simplified as appropriate.

In this modification example, an X-ray image is continuously taken during the pull-back, which is an operation of pulling the sensor 28 to the hand side, and the position where the sensor 28 appears on the corresponding X-ray image is continuously captured every time a two-dimensional image is generated, that is, every frame. As a result, it is possible to construct the curved three-dimensional image 70 in real time.

In the following description, it is assumed that i when steps S1A to S7A are executed first is “1”, i when steps S1A to S7A are executed last is “2”, and i when steps S1A to S7A are executed the second and subsequent times excluding the last is “3” or more and is incremented by 1 every time. For example, when steps S1A to S7A are executed four times in total, i has values of “1”, “3”, “4”, and “2” in order. When i is “1”, steps S3A, S4A, S6A, and S7A may be skipped. After S7A, step S1A and the subsequent steps are repeatedly executed until a certain end operation is performed, such as pressing of an end button or end of pull-back.

In S1A, the control unit 21 acquires an X-ray image imaged by the X-ray imaging device 30 when the sensor 28 exists at the i-th point Pi in a biological lumen. Specifically, the control unit 21 receives the X-ray image imaged when the sensor 28 is located at the i-th point Pi from the X-ray imaging device 30 via the communication unit 23. This X-ray image may include at least an image of the sensor 28 and does not need to include an image of the axis 27 of the catheter 26.

In S2A, the control unit 21 acquires a two-dimensional image obtained by the sensor 28 when the sensor 28 exists at the i-th point Pi. Specifically, the control unit 21 receives from the sensor 28 via the communication unit 23, reflected wave information regarding a reflected wave received by the sensor 28 when the sensor 28 exists at the first point P1. The control unit 21 generates a cross-sectional image corresponding to the first point P1 as the two-dimensional image, by imaging the cross-sectional structure of the biological lumen based on the reflected wave information received. Alternatively, the control unit 21 may receive, as the two-dimensional image from a separately provided IVUS device via the communication unit 23, a cross-sectional image generated based on the reflected wave information corresponding to the first point P1 by the IVUS device.

Step S1A may be executed before S2A, or may be executed simultaneously with S2A, or may be executed after S2A.

In S3A, the control unit 21 generates a three-dimensional image 70 by imaging the three-dimensional structure of the biological lumen based on the two-dimensional image acquired in S2A, and, if any, one or more two-dimensional images which have been acquired before S2A. Specifically, the control unit 21 generates the three-dimensional image 70 by stacking the cross-sectional image generated or received in S2A, and, if any, one or more cross-sectional images which have been generated or received before S2A.

In S4A, the control unit 21 causes the display 50 to display the three-dimensional image 70 generated in S3A with being superimposed on the X-ray image acquired in S1A.

In S5A, the control unit 21 specifies the position of the sensor 28 as the sensor position in the two-dimensional image acquired in S2A. Specifically, the control unit 21 specifies the position of the center of the two-dimensional image obtained by the sensor 28 when the sensor 28 exists at the i-th point Pi, as an i-th sensor position Vi.

In S6A, the control unit 21 sets at least a part of the moving line 74 in the three-dimensional image 70 generated in S3A. The moving line 74 is a line extending from the position of the first sensor position V1 in the three-dimensional image 70 in a shape corresponding to the shape of the axis 27 of the catheter 26 in the section from the first point P1 to the second point P2 in the biological lumen. The shape of the axis 27 of the catheter 26 in the section from the first point P1 to the second point P2 in the biological lumen is specified by using a plurality of X-ray images acquired in S1A until i becomes “2”. When i is not “2”, the shape of the axis 27 of the catheter 26 to the middle of the section from the first point P1 is specified by using the X-ray image acquired in S1A so far.

Specifically, the control unit 21 automatically detects the image of the sensor 28 from the X-ray image acquired in S1A. That is, the control unit 21 automatically detects the position where the sensor 28 appears on the X-ray image acquired in S1A. As the method of detecting the position where the sensor 28 appears, a known image recognition technique can be used. Machine learning such as deep learning may be used. The control unit 21 receives from the MDU that drives the catheter 26 via the communication unit 23, a notification of the position of the sensor 28 in the long axis direction of the catheter 26 when the sensor 28 exists at the i-th point Pi. The control unit 21 specifies the i-th sensor position Vi specified in S5A in the cross section existing at the position notified from the MDU in the three-dimensional image 70, as the position of the i-th sensor position Vi in the three-dimensional image 70. The control unit 21 sets the automatically detected position as an i-th position Li and the position of the i-th sensor position Vi in the three-dimensional image 70 as an i-th setting position of the moving line 74 and stores the positions in the storage unit 22 in association with each other. A first setting position of the moving line 74 corresponds to the position of one end of the moving line 74. A second setting position of the moving line 74 corresponds to the position of the other end of the moving line 74.

In S7A, the control unit 21 adjusts the shape of the three-dimensional image 70 generated in S3A in accordance with the shape of the axis 27 of the catheter 26 in the section from the first point P1 to the second point P2 in the biological lumen, which has been specified by using a plurality of X-ray images acquired in S1A until i becomes “2”. In a case where i is not “2”, the control unit 21 adjusts the shape of the three-dimensional image 70 generated in S3A in accordance with the shape of the axis 27 of the catheter 26 in the middle of the section from the first point P1, which has been specified by using the X-ray image acquired in S1A so far. Specifically, the control unit 21 adjusts the shape of the three-dimensional image 70 generated in S3A by arranging the sensor positions specified in S5A so far on the part of the moving line 74 set in S6A so far. More specifically, the control unit 21 adjusts the relative position and size at which the three-dimensional image 70 is displayed on the display 50 and the shape of the three-dimensional image 70 with respect to the X-ray image 80 such that each setting position of the moving line 74 stored in the storage unit 22 coincides with the position on the X-ray image 80 displayed on the screen 51, which has been already stored in the storage unit 22 in association with the setting position.

Steps S5a to S7a may be executed before S4a.

As described above, in this modification example, the control unit 21 associates the first position L1 corresponding to the first point P1 on the X-ray image imaged by the X-ray imaging device 30 when the sensor 28 exists at the first point P1 in the biological lumen, with the position of one end of the moving line 74. The control unit 21 associates the second position L2 corresponding to the second point P2 on the X-ray image imaged by the X-ray imaging device 30 when the sensor 28 exists at the second point P2 in the biological lumen, with the position of the other end of the moving line 74. The control unit 21 automatically detects the image of the sensor 28 from each X-ray image included in the plurality of X-ray images imaged by the X-ray imaging device 30 while the sensor 28 moves in the section from the first point P1 to the second point P2 in the biological lumen. The control unit 21 sets the shape of the moving line 74 based on the detected image.

Thus, according to the present embodiment, it is possible to construct the curved three-dimensional image 70 in real time. Although the corresponding X-ray image is required for each cross-sectional image, it is sufficient that at least the sensor 28 appears in each X-ray image, and the axis 27 of the catheter 26 does not need to appear.

The present disclosure is not limited to the above-described embodiment. For example, two or more blocks illustrated in the block diagram may be integrated, or one block may be divided. Instead of executing two or more steps described in the flowchart in time series according to the description, the steps may be executed in parallel or in a different order, depending on the processing capability of the device that executes each step or as necessary. In addition, modifications can be made without departing from the gist of the present disclosure. For example, in a case where only the three-dimensional image 70 matching the actual blood vessel travel is displayed on the display 50, the control unit 21 does not need to display the X-ray image 80 on the display 50 in S4.

### REFERENCE SIGNS LIST

10 Image processing system
20 Image processing apparatus
21 Control unit
22 Storage unit
23 Communication unit
24 Input unit
25 Output unit
26 Catheter
27 Axis
28 Sensor
29 Guide wire
30 X-ray imaging device
40 Input device
50 Display
51 Screen
52 First region
53 Second region
54 Slider
60 Two-dimensional image
61 Human body model image
70 Three-dimensional image
71 Human body model image
72 Cross-sectional image
73 Camera icon
74 Moving line
80 X-ray image
82 Linking line

What is claimed is:

1. An image processing apparatus that generates a three-dimensional image by imaging a three-dimensional structure of a biological lumen based on a plurality of two-dimensional images obtained in a manner that a sensor provided in a catheter inserted into the biological lumen moves in a section from a first point to a second point in the biological lumen along an axis of the catheter, the image processing apparatus comprising:
a processor configured to:
adjust a shape of the three-dimensional image in accordance with a shape of the axis of the catheter in the section, the shape of the axis of the catheter being specified by using at least one X-ray image including an image of at least a part of the catheter imaged by an X-ray imaging device that performs fluoroscopy of the biological lumen.

2. The image processing apparatus according to claim 1, wherein the processor is configured to:
specify a position of the sensor as a sensor position for each two-dimensional image included in the plurality of two-dimensional images;
set, in the three-dimensional image, a moving line extending in a shape corresponding to the shape of the axis of the catheter in the section from a position in the three-dimensional image of a sensor position specified for a two-dimensional image obtained by the sensor when the sensor exists at the first point; and
adjust a shape of the three-dimensional image by arranging each specified sensor position on the set moving line.

3. The image processing apparatus according to claim 2, wherein the processor is configured to:
associate a first position corresponding to the first point on the at least one X-ray image with a position of one end of the moving line;
associate a second position corresponding to the second point on the at least one X-ray image with a position of another end of the moving line;
set a linking line linking the first position and the second position on the at least one X-ray image; and
set a shape of the moving line according to an operation of manually changing the set shape of the linking line.

4. The image processing apparatus according to claim 2, wherein the processor is configured to:
associate a first position corresponding to the first point on the at least one X-ray image with a position of one end of the moving line;
associate a second position corresponding to the second point on the at least one X-ray image with a position of another end of the moving line; and
set a shape of the moving line to be a shape corresponding to a linking line linking the first position, the second position, and at least one position different from the first position and the second position, according to an operation of manually designating the at least one position on the at least one X-ray image.

5. The image processing apparatus according to claim 2, wherein the processor is configured to:
associate a first position corresponding to the first point on the at least one X-ray image with a position of one end of the moving line;
associate a second position corresponding to the second point on the at least one X-ray image with a position of another end of the moving line;
automatically detect an image of the axis of the catheter from the at least one X-ray image; and
set a shape of the moving line based on the detected image.

6. The image processing apparatus according to claim 2, wherein the processor is configured to:
associate a first position corresponding to the first point on the at least one X-ray image with a position of one end of the moving line;
associate a second position corresponding to the second point on the at least one X-ray image with a position of another end of the moving line;
automatically detect an image of the sensor from each X-ray image included in a plurality of X-ray images imaged by the X-ray imaging device while the sensor moves in the section; and
set a shape of the moving line based on the detected image.

7. The image processing apparatus according to claim 2, wherein the processor is configured to:
associate a first position corresponding to the first point on the at least one X-ray image with a position of one end of the moving line according to an operation of manually designating the first position when the sensor exists at the first point.

8. The image processing apparatus according to claim 7, wherein the processor is configured to:
associate a second position corresponding to the second point on the at least one X-ray image with a position of another end of the moving line according to an operation of manually designating the second position when the sensor exists at the second point.

9. The image processing apparatus according to claim 7, wherein the processor is configured to:
acquire ratio information indicating a ratio of a dimension of the at least one X-ray image with respect to a dimension of the three-dimensional image; and
associate a position separated from the first position by a distance corresponding to a distance of the moving line on the at least one X-ray image with a position of another end of the moving line as a second position corresponding to the second point, on the at least one X-ray image based on the acquired ratio information.

10. The image processing apparatus according to claim 2, wherein the processor is configured to:
automatically detect a first position corresponding to the first point on the at least one X-ray image when the sensor exists at the first point and associate the first position with a position of one end of the moving line.

11. The image processing apparatus according to claim 10, wherein the processor is configured to:
automatically detect a second position corresponding to the second point on the at least one X-ray image when the sensor exists at the second point and associate the second position with a position of another end of the moving line.

12. The image processing apparatus according to claim 2, wherein the processor is configured to:
cause a display to display the three-dimensional image with being superimposed on the at least one X-ray image.

13. The image processing apparatus according to claim 12, wherein the processor is configured to:
update the three-dimensional image according to a change in the moving line during setting of the moving line.

14. An image processing system comprising:
the image processing apparatus according to claim 1; and
a display that displays the three-dimensional image and the at least one X-ray image.

15. An image processing method of generating a three-dimensional image by imaging a three-dimensional structure of a biological lumen based on a plurality of two-dimensional images obtained in a manner that a sensor provided in a catheter inserted into the biological lumen moves in a section from a first point to a second point in the biological lumen along an axis of the catheter, the image processing method comprising:
adjusting, by a processor, a shape of the three-dimensional image in accordance with a shape of the axis of the catheter in the section, the shape of the axis of the catheter being specified by using at least one X-ray image including an image of at least a part of the catheter imaged by an X-ray imaging device that performs fluoroscopy of the biological lumen.

16. The image processing method according to claim 15, further comprising:
specifying, by the processor, a position of the sensor as a sensor position for each two-dimensional image included in the plurality of two-dimensional images;
setting, by the processor, in the three-dimensional image, a moving line extending in a shape corresponding to the shape of the axis of the catheter in the section from a position in the three-dimensional image of a sensor position specified for a two-dimensional image obtained by the sensor when the sensor exists at the first point; and
adjusting, by the processor, a shape of the three-dimensional image by arranging each specified sensor position on the set moving line.

17. The image processing method according to claim 16, further comprising:
associating, by the processor, a first position corresponding to the first point on the at least one X-ray image with a position of one end of the moving line;
associating, by the processor, a second position corresponding to the second point on the at least one X-ray image with a position of another end of the moving line;
setting, by the processor, a linking line linking the first position and the second position on the at least one X-ray image; and
setting, by the processor, a shape of the moving line according to an operation of manually changing the set shape of the linking line.

18. The image processing method according to claim 16, further comprising:
associating, by the processor, a first position corresponding to the first point on the at least one X-ray image with a position of one end of the moving line;
associating, by the processor, a second position corresponding to the second point on the at least one X-ray image with a position of another end of the moving line; and
setting, by the processor, a shape of the moving line to be a shape corresponding to a linking line linking the first position, the second position, and at least one position different from the first position and the second position, according to an operation of manually designating the at least one position on the at least one X-ray image.

19. The image processing method according to claim 16, further comprising:
associating, by the processor, a first position corresponding to the first point on the at least one X-ray image with a position of one end of the moving line;
associating, by the processor, a second position corresponding to the second point on the at least one X-ray image with a position of another end of the moving line;
automatically detecting, by the processor, an image of the axis of the catheter from the at least one X-ray image; and
setting, by the processor, a shape of the moving line based on the detected image.

20. A non-transitory computer-readable medium storing an image processing program for causing a computer to generate a three-dimensional image by imaging a three-dimensional structure of a biological lumen based on a plurality of two-dimensional images obtained in a manner that a sensor provided in a catheter inserted into the biological lumen moves in a section from a first point to a second point in the biological lumen along an axis of the catheter to execute an operation including adjusting a shape of the three-dimensional image in accordance with a shape of the axis of the catheter in the section, the shape of the axis of the catheter being specified by using at least one X-ray image including an image of at least a part of the catheter imaged by an X-ray imaging device that performs fluoroscopy of the biological lumen.
